SEQUENCE LISTING (38 sequences)

SEQ ID NO: 1 - protein, 16 aa, Homo sapiens
HFICNYGPGGNYPTWP

SEQ ID NO: 2 - RNA, 48 nt, Artificial Sequence
  Description of Artificial Sequence Synthetic consensus sequence
cayuuyauhugygayuayggnccnggnggngayuayccnacnuayccn

SEQ ID NO: 3 - protein, 266 aa, Homo sapiens
MRVTLATIAWMVSFVSNYSHTANILPDIENEDFIKDCVRIHNKFRSEVKPTASDMLYMTWDPALAQIAKAWASNCQFSHNTRLKPPHKLHPNFTSLGENIWTGSVPIFSVSSAITNWYDEIQDYDFKTRICKKVCGHYTQVVWADSYKVGCAVQFCPKVSGFDALSNGAHFICNYGPGGNYPTWPYKRGATCSACPNNDKCLDNLCVNRQRDQVKRYYSVVYPGWPIYPRNRYTSLFLIVNSVILILSVIITILVQHKYPNLVLLD

SEQ ID NO: 4 - protein, 199 aa, Homo sapiens
ANILPDIENEDFIKDCVRIHNKFRSEVKPTASDMLYMTWDPALAQIAKAWASNCQFSHNTRLKPPHKLHPNFTSLGENIWTGSVPIFSVSSAITNWYDEIQDYDFKTRICKKVCGHYTQVVWADSYKVGCAVQFCPKVSGFDALSNGAHFICNYGPGGNYPTWPYKRGATCSACPNNDKCLDNLCVNRQRDQVKRYYSV

SEQ ID NO: 5 - protein, 196 aa, Necator americanus
AEAEFGCPDNGMSEEARQKFLEMHNSLRSSVALGQAKDGAGGNAPKAAKMKTMAYDCEVEKTAMNNAKQCVFKHSQPNQRKGLGENIFMSSDSGMDKAKAAEQASKAWFGELAEKGVGQNLKLTGGLFSRGVGHYTQMVWQETVKLGCYVEACSNMCYVVCQYGPAGNMMGKDIYEKGEPCSKCENCDKEKGLCSA

SEQ ID NO: 6 - protein, 154 aa, Homo sapiens
MGKSASKQFHNEVLKAHNEYRQKHGVPPLKLCKNLNREAQQYSEALASTRILKHSPESSRGQCGENLAWASYDQTGKEVADRWYSEIKNYNFQQPGFTSGTGHFTAMVWKNTKKMGVGKASASDGSSFVVARYFPAGNVVNEGFFEENVLPPKK

SEQ ID NO: 7 - protein, 209 aa, Vespula vulgaris
AEAEFNNYCKIKCLKGGVHTACKYGSLKPNCGNKVVVSYGLTKQEKQDILKEHNDFRQKIARGLETRGNPGPQPPAKNMKNLVWNDELAYVAQVWANQCQYGHDTCRDVAKYQVGQNVALTGSTAAKYDDPVKLVKMWEDEVKDYNPKKKFSGNDFLKTGHYTQMVWANTKEVGCGSIKYIQEKWHKHYLVCNYGPSGNFKNEELYQTK

SEQ ID NO: 8 - protein, 221 aa, Trimeresurus stejnegeri
NVDFDSESPRKPEIQNEIVDLHNSLRRSVNPTASNMLRMEWYPEAADNAERWAYRCIESHSSYESRVIEGIKCGENIYMSPYPMKWTDIIHAWHDEYKDFKYGVGADPPNAVTGHYTQIVWYKSYRIGCAAAYCPSSPYSYFFVCQYCPAGNFIGKTATPYTSGTPCGDCPSDCDNGLCTNPCTRENKFTNCNTMVQQSSCQDNYMKTNCPASCFCQNKII

SEQ ID NO: 9 - protein, 8 aa, Artificial Sequence
  Description of Artificial Sequence Synthetic peptide
PLAQGGGG

SEQ ID NO: 10 - protein, 7 aa, Artificial Sequence
  Description of Artificial Sequence Synthetic peptide
GLAQGGG

SEQ ID NO: 11 - protein, 9 aa, Homo sapiens
ANILPDIEN

SEQ ID NO: 12 - DNA, 27 bp, Homo sapiens
  CDS (1)..(27)
gcaaatattttgccagatatcgaaaat

SEQ ID NO: 13 - protein, 12 aa, Homo sapiens
EVKPTASDMLYM

SEQ ID NO: 14 - DNA, 36 bp, Homo sapiens
  CDS (1)..(36)
gaggtgaaaccaacagccagtgatatgctatacatg

SEQ ID NO: 15 - protein, 24 aa, Homo sapiens
SNCQFSHNTRLKPPHKLHPNFTSL

SEQ ID NO: 16 - DNA, 72 bp, Homo sapiens
  CDS (1)..(72)
agcaattgccagttttcacataatacacggctgaagccaccccacaagctgcacccaaacttcacttcactg

SEQ ID NO: 17 - protein, 5 aa, Homo sapiens
VPIFS

SEQ ID NO: 18 - DNA, 15 bp, Homo sapiens
  CDS (1)..(15)
gtgcccattttttct

SEQ ID NO: 19 - protein, 4 aa, Homo sapiens
FKTR

SEQ ID NO: 20 - DNA, 12 bp, Homo sapiens
  CDS (1)..(12)
ttcaagactcgg

SEQ ID NO: 21 - protein, 11 aa, Homo sapiens
PKVSGFDALSN

SEQ ID NO: 22 - DNA, 33 bp, Homo sapiens
  CDS (1)..(33)
cctaaagtttctggctttgacgctctttccaat

SEQ ID NO: 23 - protein, 24 aa, Homo sapiens
GPGGNYPTWPYKRGATCSACPNND

SEQ ID NO: 24 - DNA, 72 bp, Homo sapiens
  CDS (1)..(72)
ggaccaggagggaattacccaacttggccatataagagaggagccacctgcagtgcctgccccaataatgac

SEQ ID NO: 25 - protein, 3 aa, Homo sapiens
LDN

SEQ ID NO: 26 - DNA, 9 bp, Homo sapiens
  CDS (1)..(9)
ttggacaat

SEQ ID NO: 27 - protein, 21 aa, Homo sapiens
CQFSHNTRLKPPHKLHPNFTS

SEQ ID NO: 28 - protein, 5 aa, Homo sapiens
KRYYS

SEQ ID NO: 29 - protein, 6 aa, Artificial Sequence
  Description of Artificial Sequence Synthetic peptide
EAEAEF

SEQ ID NO: 30 - protein, 11 aa, Homo sapiens
GHYTQVVWADS

SEQ ID NO: 31 - protein, 12 aa, Homo sapiens
HFICNYGPGGNY

SEQ ID NO: 32 - protein, 16 aa, Homo sapiens
SNGAHFICNYGPGGNY

SEQ ID NO: 33 - protein, 16 aa, Homo sapiens
NYGPGGNYPTWPYKRG

SEQ ID NO: 34 - DNA, 20 bp, Artificial Sequence
  Description of Artificial Sequence Synthetic primer
gatggtttcttttgtctcca

SEQ ID NO: 35 - DNA, 20 bp, Artificial Sequence
  Description of Artificial Sequence Synthetic primer
gttaacacagagattgtcca

SEQ ID NO: 36 - DNA, 21 bp, Artificial Sequence
  Description of Artificial Sequence Synthetic primer
gctcgtcgtcgacaacggctc

SEQ ID NO: 37 - DNA, 25 bp, Artificial Sequence
  Description of Artificial Sequence Synthetic primer
caaacatgatctgggtcatcttctc

SEQ ID NO: 38 - protein, 21 aa, Artificial Sequence
  Description of Artificial Sequence Synthetic peptide
CQFSHNTRLKPPHKLHPNFTS

(19) United States
(12) Patent Application Publication
(10) Pub. No.: US 2013/0156777 A1
(43) Pub. Date: Jun. 20, 2013

(54) GLIPR1 INHIBITORS AND THERAPEUTIC USES THEREOF

(71) Applicants: Raymond A. Koski, Old Lyme, CT (US); Nathalie Bonafe, New Haven, CT (US); Oluwatoyin A. Asojo, Houston, TX (US)

(72) Inventors: Raymond A. Koski, Old Lyme, CT (US); Nathalie Bonafe, New Haven, CT (US); Oluwatoyin A. Asojo, Houston, TX (US)

(21) Appl. No.: 13/606,866

(22) Filed: Sep. 7, 2012

Related U.S. Application Data
(60) Provisional application No. 61/533,564, filed on Sep. 12, 2011.

(51) Int. Cl. C07K 16/30

(52) U.S. Cl. CPC C07K 16/3053
USPC 424/139.1; 530/387.9; 530/391.3; 530/391.7; 435/375

(57) ABSTRACT

Featured herein are unique epitopes of human glioma pathogenesis-related protein 1 (hGLIPR-1), nucleic acids encoding the same; inhibitors of the nucleic acids and polypeptides; as well as methods for treating certain cancers and viral infections in a subject by administering to the subject an effective amount of a GLIPR1 inhibitor.

### RELATED APPLICATIONS

The present application is a non-provisional patent application claiming priority to U.S. Provisional Application No. 61/533,564 filed Sep. 12, 2011, the contents of which are incorporated by reference herein in their entireties.

### BACKGROUND OF THE INVENTION

Gliomas are malignant tumors that arise from glial cells in the central nervous system. They are among the deadliest of human cancers. In industrialized countries, glioblastoma multiforme (GBM) is the most common malignant primary brain tumor. These tumors are notorious for their aggressive and highly invasive behavior and their genetic heterogeneity, explaining in part why the majority of successful preclinical studies have failed to provide significant clinical results. Poor outcome is due to tumor recurrence that happens 100% of the time. While conventional anticancer therapies may decrease the bulk of the tumor mass, mainly comprised of differentiated cancer cells, they are unlikely to result in long-term remissions if the subsets of cancer-initiating cells, i.e., cancer stem cells, that maintain the tumor growth are not targeted. Tumors, like adult tissues, arise from cells that exhibit the ability to self-renew as well as give rise to differentiated tissue cells. Small populations of stem-like cells exist in the glioma tumor mass and harbor drug resistance and low radiosensitivity, and thus result in tumor recurrence after various treatments.

The cysteine-rich secretory proteins, antigen 5, and pathogenesis-related 1 proteins (CAP) superfamily members have been described for various eukaryotic organisms, including plants and animals (vertebrates and invertebrates). Capable of surviving harsh environments, resistant to proteolysis, these CAP proteins are often found in cells capable of migration, infiltration, invasion or virulence (neutrophils, sperm, cancer cells, fibroblast, Candida). In mammals, the majority of the CAP family members is expressed in the reproductive tract and immune tissues or in deregulated cancers. Up-regulation of certain CAP proteins has been associated with human pathogenesis/chronic diseases, such as glioblastoma, prostate cancer, kidney fibrosis, chronic pancreatitis and cardiac disease. All members share a structurally unique α-β-α sandwich core and a cluster of conserved histidine and glutamic acid residues that have been proposed to form an active site or a putative Zn²⁺ binding site (Gibbs, G. M. et al., (2008) Endocr Rev 29, 865-97), but their molecular functions are generally unclear. One member, in particular, the human Glioma Pathogenesis-Related Protein 1 (GLIPR1, RTVP-1) was found over-expressed in glial tumors and glioma-derived cell lines, and expression of GLIPR1/RTVP-1 was barely detected using semi-quantitative RT-PCR in normal brains (Murphy, E. V., et al., (1995) Gene 159, 131-5; Rosenzweig, T., et al., (2006) Cancer Res 66, 4139-48). Using siRNA knockdown and overexpression studies in glioma cells lines, Rosenzweig et al. (Rosenzweig, T., et al., (2006) Cancer Res 66, 4139-48) demonstrated a direct correlation between GLIPR1 overexpression and increased glioma cell growth, survival and invasion (See also U.S. Patent Publication No. 20060216731). GLIPR1 siRNA was also found to inhibit HIV-1 replication in cultured cells (Capalbo, G., et. al., (2010) Retrovirology 7:26 and U.S. Patent Publication No. 20090233985). Overexpression of GLIPR1 rendered glioma cells more resistant to apoptosis, resulting in higher levels of Bcl2, and lower levels of phosphorylated c-Jun kinase (JNK). While GLIPR1 has a tumor-promoting role in glioma cells, it is proposed to act as a tumor suppressor in prostate cells (Gibbs, G. M. et al., (2008) Endocr Rev 29, 865-97, Ren, C., et al., (2002) Mol Cell Biol 22, 3345-57; Ren, C., et al., (2004) Cancer Res 64, 969-76; and Thompson, T. C. (2010) Yonsei Med J 51, 479-83. See also U.S. Pat. Nos. 7,723,475; 7,645,452; 7,601,806 and U.S. Patent Publication No. 20050187153). However, it is not uncommon for proteins, in particular membrane proteins associated with lipid rafts, to function in a tissue-specific and microenvironment-dependent manner (Burgermeister, E., et al., (2008) Cancer Lett 268, 187-201).

New treatments for gliomas and other cancers, are needed.

### SUMMARY OF THE INVENTION

In one aspect, the invention features unique epitopes of the human glioma pathogenesis-related protein 1 (GLIPR1). In one embodiment, the epitope comprises the following polypeptide sequence: 170-HFICNYGPGGNYPTWP-185 (SEQ ID NO: 1). This unique site is completely abrogated by main chains in other CAP structures, whereas in GLIPR1 (native and soluble), this motif is solvent-accessible and contains an epitope recognized by antibodies elicited by immunization with sGLIPR1. In another embodiment, the epitope comprises a polypeptide sequences selected from the group consisting of: ANILPDIEN, SEQ ID NO: 11; EVKPTASDMLYM, SEQ ID NO: 13; SNCQFSHNTRLKPPHKLHPNFTSL, SEQ ID NO: 15; VPIFS, SEQ ID NO: 17; FKTR, SEQ ID NO: 19; PKVSGFDALSN, SEQ ID NO: 21; GPGGNYPTWPYKRGATCSACPNND, SEQ ID NO: 23 and LDN, SEQ ID NO: 25.

In another aspect, the invention features nucleic acids encoding the unique GLIPR-1 epitopes. In one embodiment, the nucleic acid is represented by the following consensus sequence: CAYUUYAUHUGYGAYUAYGGNCCNGGNGGNGAYUAYCCNACNUAYCCN (SEQ ID NO: 2). In other embodiments, the consensus sequence is represented by the following nucleic acid sequences: (encoding ANILPDIEN), SEQ ID NO: 12; (encoding EVKPTASDMLYM) SEQ ID NO: 14; (encoding SNCQFSHNTRLKPPHKLHPNFTSL), SEQ ID NO: 16; (encoding VPIFS), SEQ ID NO: 18; (encoding FKTR), SEQ ID NO: 20; (encoding PKVSGFDALSN), SEQ ID NO: 22; (encoding GPGGNYPTWPYKRGATCSACPNND), SEQ ID NO: 24 and (encoding LDN), SEQ ID NO: 26.

In another aspect, the invention features molecules that can bind to GLIPR1 epitopes. In certain embodiments, the molecules bind to SEQ ID NOs: 1, 11, 13, 15, 17, 19, 21, 23 and 25. In certain embodiments, the molecule is selected from the group consisting of a protein (e.g., an antibody), a peptide, a nucleic acid and a small molecule. In certain embodiments, the molecule binds GLIPR1 on the surface of cells. In other embodiments, the molecule binds GLIPR1 in lipid rafts on the surface of cells. Binding molecules may be inhibitory or non-inhibitory. In certain embodiments, binding molecules reduce or inhibit cancer cell matrix metalloproteinase-2 (MMP-2) secretion, proliferation, invasion, migration and/or neurosphere formation. In other embodiments, the binding molecule down regulates cell surface GLIPR1 and/or induces cleavage of the AKT kinase.

In another aspect, the invention features pharmaceutical compositions comprising a GLIPR1 binding molecule and a pharmaceutically acceptable carrier.

In yet a further aspect, the GLIPR1 binding molecule is further conjugated to a diagnostic or therapeutic agent (e.g., an anticancer or antiviral agent).

In a further aspect, the invention features methods for inhibiting GLIPR-1 activity in a subject comprising administering to the subject an effective amount of a GLIPR-1 inhibitor alone or in conjunction with a pharmaceutical carrier. In certain embodiments, the method treats or prevents the development of a cancer in the subject. In certain embodiments, the cancer is a glioma. In other embodiments, the method treats or prevents the development of a viral infection in the subject.

Further features and advantages will now be disclosed in conjunction with the following Detailed Description and Claims.

### BRIEF DESCRIPTION OF THE FIGURES

FIG. 1 is a comparison of the three dimensional structure of soluble GLIPR1 (sGLIPR1) with representative CAP structures. The top row shows a ribbon diagram of sGLIPR1 that reveals a conserved core CAP domain similar to that observed in representative CAP structures GAPR-1, stecrisp, Ves-v5 and Na-ASP-2. This core α-β-α sandwich is formed by the three core β strands flanked by the labelled helices. Arrows indicate the location of loops in sGLIPR1 that are longer than in the other structures. Overall the structure of sGLIPR1 is more similar to Na-ASP-2 than the others. The bottom row reveals that surface charge distribution is different for these representative CAP structures. Negatively charged regions are lightly shaded and positively charged regions are represented with darker shading. The same view is shown for all images.

FIG. 2 is a primary sequence alignment of sGLIPR1 with representative CAP protein sequences: GLIPR1 (SEQ ID NO: 3); sGLIPR1 (SEQ ID NO: 4); Na-Asp-2 (SEQ ID NO: 5); GAPR-1 (SEQ ID NO: 6); Ves-v5 (SEQ ID NO: 7) and stecrisp (SEQ ID NO: 8). Helices and strand regions of the core CAP domain are well conserved. The highest variability is in the loop regions. Also identified are the cysteine residues involved in disulfide bonds as well as location of the signature prosite CRISP1 and CRISP2 motifs.

FIG. 3 is a comparison of CAP protein central cavities. (a) Key residues in putative binding cavity of sGLIPR1 without Zn²⁺ align well with other CAP structures, and appear capable of forming the same interaction to Zn²⁺ as in both snake venom CRISP structures. The superimposed CAP structures include sGLIPR1, Na-ASP-2, Ves-v5, GAPR1 and stecrisp, natrin with Zn²⁺, pseudecin with Zn²⁺. Also shown are the Zn²⁺ from both structures which are in the same position and the complexed water molecule. (b) Putative binding cavity of structure of sGLIPR1 soaked with zinc chloride reveals a bound Zn²⁺ ion coordinated to two His and a water molecule. The Refmac-5 generated 2Fo-Fc electron-density map is contoured at 1.5 sigma.

FIG. 4 is a comparison of the three dimensional structure of soluble GLIPR1 (sGLIPR1) with representative CAP structures. GLIPR1 has a unique surface-accessible region. Surface representation of sGLIPR1, with the region corresponding to a linear antibody epitope (SEQ ID NO: 1) mapped in darker shading, reveals an exposed cleft indicated by an arrow. The cleft is blocked by main chain residues forming surfaces in the structure of Na-ASP-2, stecrisp, Ves-v5 and GAPR-1 (as shown in the superimposed structures). The extent to which main chains of each of the representative CAP structures obscure the exposed cleft of sGLIPR1 is also shown in the ribbon representations using the same orientation. The cleft is blocked by a differently situated, flexible loops in the structures of Na-ASP-2, stecrisp, Ves-v5 and GAPR-1.

FIG. 5 shows antibody binding to overlapping peptides spanning the sGLIPR1 sequence. Antibody preparations, including anti-sGLIPR1 (a) and anti-native GLIPR1 (b) that inhibit U87 glioma cell growth, and anti-GLIPR1₇₅₋₉₅ (c) and normal rabbit IgGs (d) negative control antibodies that do not inhibit U87 cell growth, were tested on a total of 47 overlapping 16-mer GLIPR1 peptides (X axis). Antibody binding to peptide 38 (SEQ ID NO: 1) is highlighted (black) in (a) and (b). Two other peptides, PLAQGGGG (SEQ ID NO: 9) and GLAQGGG (SEQ ID NO: 10), were tested with anti-sperm whale myoglobin antibody, and served as the manufacturer's positive and negative controls, respectively (white).

### DETAILED DESCRIPTION

### 1. General

Human glioma pathogenesis-related protein 1 (GLIPR1), a member of the CAP (cysteine-rich secretory protein, antigen 5, pathogenesis related-1) superfamily, is composed of a signal peptide, to direct its secretion, a conserved cysteine-rich CAP domain, and a transmembrane domain.

Human GLIPR1 (NCBI accession No. NP_{—}006842) is naturally synthesized as a 255-amino acid precursor with a signal peptide and a transmembrane domain that localizes the mature protein to the cell membrane in glioblastoma cells. The first 21 residues make up the signal peptide, while C-terminal residues form the predicted membrane-spanning domain. The recombinant sGLIPR1 protein consists of amino acids 22-220 of human GLIPR1 with a Pichia pastoris signal peptide and the linker amino-acid sequence EAEAEF added to the N-terminus by cloning procedures (Bonafé et al., (2010) Acta Crystallogr F66:1487-14899).

The coordinates and structure factors for the soluble domain of the human GLIPR1 protein, sGLIPR1, solved in the orthorhombic space group P2₁2₁2, has been deposited with the Research Collaboratory Structural Bioinformatics Protein Data Bank (RCSBPDB) under accession number 3q2u). The native structure was refined to 1.85 Å resolution, resulting in the discovery that of the 193 residues in the sGLIPR1 model, 54.4% are in loop or turn regions. These are extensive flexible loop/turn regions and unique charge distributions that were not observed in any of the previously reported CAP protein structures. Sequence alignment using secondary-structure elements of sGLIPR1 using ESPript (Gouet, P., et al., (2003) Nucleic Acids Res. 31, 3320-3323) suggests that sGLIPR1 is a suitable structural model for the CAP domains of other members of the GLIPR family.

The structure of the Zn²⁺ complex (RCSBPDP accession no. 3q2r) was refined to 2.2 Å, revealing that the putative binding cavity coordinates Zn²⁺ and may be involved in a Zn²⁺ dependent mechanism of inflammatory modulation.

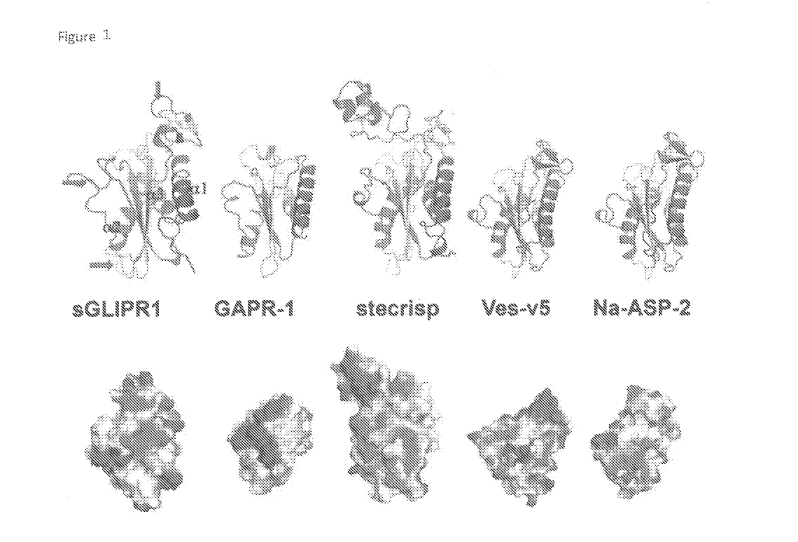

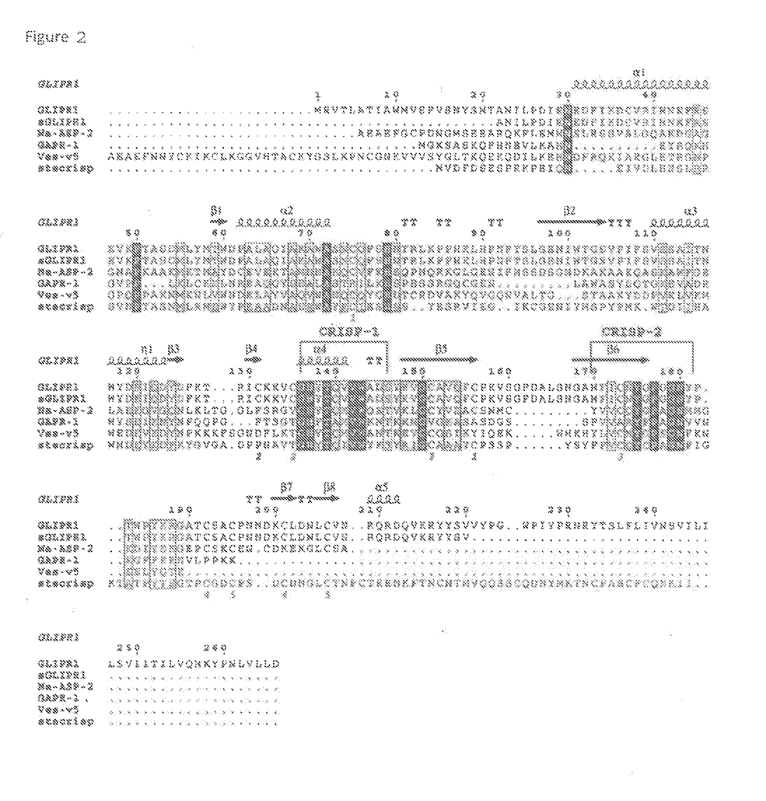

Both structures reveal a core region conserved amongst CAP proteins, and flexible surface loops unique to GLIPR1. These loop/turn regions and unique charge distributions, which are mostly on the surface of the structure, include but are not limited to the following: Ala22-Asn30 (ANILPDIEN, SEQ ID NO: 11, which is encoded by the consensus sequence shown as SEQ ID NO: 12), Glu47-Met58 (EVKPTASDMLYM, SEQ ID NO: 13, which is encoded by the consensus sequence shown as SEQ ID NO: 14), Ser73-Leu96 (SNCQFSHNTRLKPPHKLHPNFTSL, SEQ ID NO: 15, which is encoded by the consensus sequence shown as SEQ ID NO: 16), Val105-Ser109 (VPIFS, SEQ ID NO: 17, which is encoded by the consensus sequence shown as SEQ ID NO: 18), Phe126-Arg129 (FKTR, SEQ ID NO: 19, which is encoded by the consensus sequence shown as SEQ ID NO: 20), Pro157-Asn167 (PKVSGFDALSN, SEQ ID NO: 21, which is encoded by the consensus sequence shown as SEQ ID NO: 22), Gly176-Asp199 (GPGGNYPTWPYKRGATCSACPNND, SEQ ID NO: 23, which is encoded by the consensus sequence shown as SEQ ID NO: 24) and Leu202-Asn204 (LDN, SEQ ID NO: 25, which is encoded by the consensus sequence shown as SEQ ID NO: 26) (See FIGS. 1 and 2). The unique lengths and orientations of these loop/turn regions are likely to explain why phasing by molecular replacement (MR) failed when the loops were not removed from the initial search model. The lengths of the helices and strands, and the orientations and locations of the loops in sGLIPR1 could not be predicted based on any previous CAP structures.

Among the surface loops is a potential binding site adjacent to the conserved CAP2 motif (170-HFICNYGPGGNYPTWP-185, SEQ ID NO: 1). This unique site is completely abrogated by main chains in other CAP structures, whereas in GLIPR1 (native and soluble), this motif is solvent-accessible and contains an epitope recognized by antibodies elicited by immunization with sGLIPR1. Examples 1 and 2 describe antibodies of GLIPR1 and their ability to inhibit glioblastoma cell proliferation.

### 2. Definitions

For convenience, the meaning of certain terms and phrases employed in the specification, examples, and appended claims are provided below. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

“Antagonist” or “inhibitor” refers to a molecule that suppresses or inhibits at least one bioactivity of a protein. For example, a GLIPR1 antagonist may be a compound which reduces glioma cell migration, invasion, proliferation and/or neurosphere formation.

As used herein the term “antibody” refers to an immunoglobulin and any antigen-binding portion of an immunoglobulin (e.g., IgG, IgD, IgA, IgM and IgE) i.e., a polypeptide that contains an antigen-binding site, which specifically binds (“immunoreacts with”) an antigen. Antibodies can comprise at least one heavy (H) chain and at least one light (L) chain interconnected by at least one disulfide bond. The term “V_{H}” refers to a heavy chain variable region of an antibody. The term “V_{L}” refers to a light chain variable region of an antibody. In exemplary embodiments, the term “antibody” specifically covers monoclonal and polyclonal antibodies. A “polyclonal antibody” refers to an antibody, which has been derived from the sera of animals immunized with an antigen or antigens. A “monoclonal antibody” refers to an antibody produced by a single clone of hybridoma cells. Techniques for generating monoclonal antibodies include, but are not limited to, the hybridoma technique (see Kohler & Milstein (1975) Nature 256:495-497); the trioma technique; the human B-cell hybridoma technique (see Kozbor, et al. (1983) Immunol. Today 4:72), the EBV hybridoma technique (see Cole, et al., 1985 In: Monoclonal Antibodies and Cancer Therapy, Alan R. Liss, Inc., pp. 77-96) and phage display.

“Chimeric antibodies” are encoded by immunoglobulin genes that have been genetically engineered so that the light and heavy chain genes are composed of immunoglobulin gene segments belonging to different species. For example, substantial portions of the variable (V) segments of the genes from a mouse monoclonal antibody, e.g., obtained as described herein, may be joined to substantial portions of human constant (C) segments. Such a chimeric antibody is likely to be less antigenic to a human than a mouse monoclonal antibody.

As used herein, the term “humanized antibody” (HuAb) refers to a chimeric antibody with a framework region substantially identical (i.e., at least 85%) to a human framework, having CDRs from a non-human antibody, and in which any constant region has at least about 85-90%, and preferably about 95% polypeptide sequence identity to a human immunoglobulin constant region. All parts of such a HuAb, except possibly the CDRs, are substantially identical to corresponding parts of one or more native human immunoglobulin sequences. The term “framework region” as used herein, refers to those portions of immunoglobulin light and heavy chain variable regions that are relatively conserved (i.e., other than the CDRs) among different immunoglobulins in a single species, as defined by Kabat, et al. (1987) Sequences of Proteins of Immunologic Interest, 4^{th} Ed., US Dept. Health and Human Services. Human constant region DNA sequences can be isolated in accordance with well known procedures from a variety of human cells, but preferably from immortalized B cells. The variable regions or CDRs for producing humanized antibodies may be derived from monoclonal antibodies capable of binding to the antigen, and will be produced in any convenient mammalian source, including mice, rats, rabbits, or other vertebrates.

“Composite human antibodies” refer to antibodies, which have been modified to remove one or more T-cell epitopes. See e.g., US 2008/0206239.

The term “antibody” also encompasses antibody fragments. Examples of antibody fragments include Fab, Fab′, Fab′-SH, F(ab′)₂, and Fv fragments; diabodies and any antibody fragment that has a primary structure consisting of one uninterrupted sequence of contiguous amino acid residues, including without limitation: single-chain Fv (scFv) molecules, single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific or multivalent structures formed from antibody fragments. In an antibody fragment comprising one or more heavy chains, the heavy chain(s) can contain any constant domain sequence (e.g., CH1 in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s). Suitable leucine zipper sequences include the jun and fos leucine zippers taught by Kostelney et al., (1992) J. Immunol., 148: 1547-1553 and the GCN4 leucine zipper described in U.S. Pat. No. 6,468,532. Fab and F(ab′)₂ fragments lack the Fc fragment of intact antibody and are typically produced by proteolytic cleavage, using enzymes such as papain (to produce Fab fragments) or pepsin (to produce F(ab′)₂ fragments).

An antibody “specifically binds” to an antigen or an epitope of an antigen if the antibody binds preferably to the antigen over most other antigens. For example, the antibody may have less than about 50%, 20%, 10%, 5%, 1% or 0.1% cross-reactivity toward one or more other epitope.

The term “conservative substitution” refers to a change between amino acids of broadly similar molecular properties. For example, interchanges within the aliphatic group alanine, valine, leucine and isoleucine can be considered as conservative. Sometimes substitution of glycine for one of these can also be considered conservative. Other conservative interchanges include those within the aliphatic group aspartate and glutamate; within the amide group asparagine and glutamine; within the hydroxyl group serine and threonine; within the aromatic group phenylalanine, tyrosine and tryptophan; within the basic group lysine, arginine and histidine; and within the sulfur-containing group methionine and cysteine. Sometimes substitution within the group methionine and leucine can also be considered conservative. Preferred conservative substitution groups are aspartate-glutamate; asparagine-glutamine; valine-leucine-isoleucine; alanine-valine; valine-leucine-isoleucine-methionine; phenylalanine-tyrosine; phenylalanine-tyrosine-tryptophan; lysine-arginine; and histidine-lysine-arginine.

An “effective amount” is an amount sufficient to produce a beneficial or desired clinical result upon treatment. An effective amount can be administered to a patient in one or more doses. In terms of treatment, an effective amount is an amount that is sufficient to decrease infection or tumor burden in a patient. Several factors are typically taken into account when determining an appropriate dosage to achieve an effective amount. These factors include age, sex and weight of the patient, the condition being treated, the severity of the condition and the form and effective concentration of the agent administered.

The term “epitope” refers to that region of an antigen to which an antibody or other binding molecule binds preferentially and specifically.

“Equivalent” when used to describe nucleic acids or nucleotide sequences refers to nucleotide sequences encoding functionally equivalent polypeptides. Equivalent nucleotide sequences will include sequences that differ by one or more nucleotide substitution, addition or deletion, such as an allelic variant; and will, therefore, include sequences that differ due to the degeneracy of the genetic code. For example, nucleic acid variants may include those produced by nucleotide substitutions, deletions, or additions. The substitutions, deletions, or additions may involve one or more nucleotides. The variants may be altered in coding regions, non-coding regions, or both. Alterations in the coding regions may produce conservative or non-conservative amino acid substitutions, deletions or additions.

“Glioma” refers to a tumor of the central nervous system, including astrocytomas, ependymal tumors, glioblastoma multiforme and primitive neuroectodermal tumors.

“Homology” or alternatively “identity” refers to sequence similarity between two peptides or between two nucleic acid molecules. Homology may be determined by comparing a position in each sequence, which may be aligned for purposes of comparison. When a position in the compared sequence is occupied by the same base or amino acid, then the molecules are homologous at that position. A degree of homology between sequences is a function of the number of matching or homologous positions shared by the sequences. The term “percent identical” refers to sequence identity between two amino acid sequences or between two nucleotide sequences. Identity may be determined by comparing a position in each sequence, which may be aligned for purposes of comparison. When an equivalent position in the compared sequences is occupied by the same base or amino acid, then the molecules are identical at that position; when the equivalent site is occupied by the same or a similar amino acid residue (e.g., similar in steric and/or electronic nature), then the molecules may be referred to as homologous (similar) at that position. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and may be used with, e.g., default settings. ENTREZ is available through the National Center for Biotechnology Information, National Library of Medicine, National Institutes of Health, Bethesda, Md. In one embodiment, the percent identity of two sequences may be determined by the GCG program with a gap weight of 1, e.g., each amino acid gap is weighted as if it were a single amino acid or nucleotide mismatch between the two sequences. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method may be utilized to align sequences. An alternative search strategy uses MPSRCH software, which runs on a MASPAR computer. MPSRCH uses a Smith-Waterman algorithm to score sequences on a massively parallel computer. This approach improves the ability to pick up distantly related matches, and is especially tolerant of small gaps and nucleotide sequence errors. Nucleic acid-encoded amino acid sequences may be used to search both protein and DNA databases. Databases with individual sequences are described in Methods in Enzymology, ed. Doolittle, supra. Databases include Genbank, EMBL, and DNA Database of Japan (DDBJ).

The terms “polynucleotide”, and “nucleic acid” are used interchangeably to refer to a polymeric form of nucleotides of any length, either deoxyribonucleotides or ribonucleotides, or analogs thereof. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, antisense nucleic acids, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term “recombinant” polynucleotide means a polynucleotide of genomic, cDNA, semisynthetic, or synthetic origin, which either does not occur in nature or is linked to another polynucleotide in a non-natural arrangement. An “oligonucleotide” refers to a single stranded polynucleotide having less than about 100 nucleotides, less than about, e.g., 75, 50, 25, or 10 nucleotides.

A “polypeptide” or peptide refers to a chain of amino acids linked together by peptide bonds. Polypeptides or peptides typically have a molecular weight of less than about 10,000. Larger polypeptides are typically referred to as proteins. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including glycine and both the D or L optical isomers, and amino acid analogs and peptidomimetics.

A “small molecule” refers to a compound, which has a molecular weight of less than about 5 kD, less than about 2.5 kD, less than about 1.5 kD, or less than about 0.9 kD. Small molecules may be, for example, nucleic acids, peptides, polypeptides, peptide nucleic acids, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays of the invention. The term “small organic molecule” refers to a small molecule that is often identified as being an organic or medicinal compound, and does not include molecules that are exclusively nucleic acids, peptides or polypeptides.

“Specifically hybridizes” refers to detectable and specific nucleic acid binding. Polynucleotides, oligonucleotides and nucleic acids selectively hybridize to nucleic acid strands under hybridization and wash conditions that minimize appreciable amounts of detectable binding to nonspecific nucleic acids. Stringent conditions may be used to achieve selective hybridization conditions as known in the art and discussed herein. Generally, the nucleic acid sequence homology between two polynucleotides, oligonucleotides, or nucleic acids will be at least 30%, 40%, 50%, 60%, 70%, 80%, 85%, 90%, 95%, 98%, 99%, or more. In certain instances, hybridization and washing conditions are performed under stringent conditions according to conventional hybridization procedures and as described further herein.

“Stringent conditions” or “stringent hybridization conditions” refer to conditions, which promote specific hybridization between two complementary polynucleotide strands, so as to form a duplex. Stringent conditions may be selected to be about 5° C. lower than the thermal melting point (Tm) for a given polynucleotide duplex at a defined ionic strength and pH. The length of the complementary polynucleotide strands and their GC content will determine the Tm of the duplex, and thus the hybridization conditions necessary for obtaining a desired specificity of hybridization. The Tm is the temperature (under defined ionic strength and pH) at which 50% of a polynucleotide sequence hybridizes to a perfectly matched complementary strand. In certain cases it may be desirable to increase the stringency of the hybridization conditions to be about equal to the Tm for a particular duplex.

A variety of techniques for estimating the Tm are available. Typically, G-C base pairs in a duplex are estimated to contribute about 3° C. to the Tm, while A-T base pairs are estimated to contribute about 2° C., up to a theoretical maximum of about 80-100° C. However, more sophisticated models of Tm are available in which G-C stacking interactions, solvent effects, the desired assay temperature and the like are taken into account. For example, probes can be designed to have a dissociation temperature (Td) of approximately 60° C., using the formula: Td=(((((3×#GC)+(2×#AT))×37)−562)/#bp)−5; where #GC, #AT, and #bp are the number of guanine-cytosine base pairs, the number of adenine-thymine base pairs, and the number of total base pairs, respectively, involved in the formation of the duplex.

Hybridization may be carried out in 5×SSC, 4×SSC, 3×SSC, 2×SSC, 1×SSC or 0.2×SSC for at least about 1 hour, 2 hours, 5 hours, 12 hours, or 24 hours. The temperature of the hybridization may be increased to adjust the stringency of the reaction, for example, from about 25° C. (room temperature), to about 45° C., 50° C., 55° C., 60° C., or 65° C. The hybridization reaction may also include another agent affecting the stringency, for example, hybridization conducted in the presence of 50% formamide increases the stringency of hybridization at a defined temperature.

The hybridization reaction may be followed by a single wash step, or two or more wash steps, which may be at the same or a different salinity and temperature. For example, the temperature of the wash may be increased to adjust the stringency from about 25° C. (room temperature), to about 45° C., 50° C., 55° C., 60° C., 65° C., or higher. The wash step may be conducted in the presence of a detergent, e.g., 0.1 or 0.2% SDS. For example, hybridization may be followed by two wash steps at 65° C. each for about 20 minutes in 2×SSC, 0.1% SDS, and optionally two additional wash steps at 65° C. each for about 20 minutes in 0.2×SSC, 0.1% SDS.

Exemplary stringent hybridization conditions include overnight hybridization at 65° C. in a solution comprising, or consisting of, 50% formamide, 10×Denhardt (0.2% Ficoll, 0.2% Polyvinylpyrrolidone, 0.2% bovine serum albumin) and 200 μg/mL of denatured carrier DNA, e.g., sheared salmon sperm DNA, followed by two wash steps at 65° C. each for about 20 minutes in 2×SSC, 0.1% SDS, and two wash steps at 65° C. each for about 20 minutes in 0.2×SSC, 0.1% SDS.

Hybridization may consist of hybridizing two nucleic acids in solution, or a nucleic acid in solution to a nucleic acid attached to a solid support, e.g., a filter. When one nucleic acid is on a solid support, a prehybridization step may be conducted prior to hybridization. Prehybridization may be carried out for at least about 1 hour, 3 hours or 10 hours in the same solution and at the same temperature as the hybridization solution (without the complementary polynucleotide strand).

Appropriate stringency conditions are known to those skilled in the art or may be determined experimentally by the skilled artisan. See, for example, Current Protocols in Molecular Biology, John Wiley & Sons, N.Y. (1989), 6.3.1-12.3.6; Sambrook et al., 1989, Molecular Cloning, A Laboratory Manual, Cold Spring Harbor Press, N.Y.; S. Agrawal (ed.) Methods in Molecular Biology, volume 20; Tijssen (1993) Laboratory Techniques in biochemistry and molecular biology-hybridization with nucleic acid probes, e.g., part I chapter 2 “Overview of principles of hybridization and the strategy of nucleic acid probe assays”, Elsevier, N.Y.; and Tibanyenda, N. et al., Eur. J. Biochem. 139:19 (1984) and Ebel, S. et al., Biochem. 31:12083 (1992).

A nucleic acid or polypeptide is “substantially homologous” to another, refers to sequences which are substantially identical to or similar in sequence with each other, giving rise to a homology of conformation and thus to retention, to a useful degree, of one or more biological (including immunological) activities. The term is not intended to imply a common evolution of the sequences.

A “subject” refers to a male or female mammal, including a human.

A “therapeutic agent” or “therapeutic” is a molecule, which is biologically, physiologically, or pharmacologically active when administered to a subject.

A “vector” is a self-replicating nucleic acid molecule that transfers an inserted nucleic acid molecule into and/or between host cells. The term includes vectors that function primarily for insertion of a nucleic acid molecule into a cell, replication of vectors that function primarily for the replication of nucleic acid, and expression vectors that function for transcription and/or translation of the DNA or RNA. Also included are vectors that provide more than one of the above functions. As used herein, “expression vectors” are defined as polynucleotides which, when introduced into an appropriate host cell, can be transcribed and translated into a polypeptide(s). An “expression system” usually connotes a suitable host cell comprised of an expression vector that can function to yield a desired expression product.

A “virus” is an art recognized term that refers to a non-cellular biological entity, which lacks metabolic machinery of its own and reproduces by using that of a host cell. Viruses comprise a molecule of nucleic acid (DNA or RNA) and can have an envelope or not. An “enveloped virus” refers to a virus that further comprises a lipid bilayer, which contains viral glycoproteins derived from a host cell membrane. In an enveloped virus, viral proteins that mediate attachment and penetration into the host cell are found in the envelope. Examples of enveloped viruses include influenza, both human and avian, human immunodeficiency virus (HIV), sudden acute respiratory syndrome (SARS) virus, human papilloma virus (HPV), herpes simplex virus (HSV), dengue virus and other flaviviruses, such as for example, yellow fever, West Nile, and tick-borne encephalitis viruses. A “non-enveloped virus” refers to a virus, which lacks a lipid bilayer. In non-enveloped viruses, the capsid mediates attachment to and penetration into host cells. Examples of non-enveloped viruses include Norwalk virus, hepatitis B virus, polio virus, and rhinoviruses.

### 3. GLIPR1 Inhibitors

Based on the identification of surface loops and turns of folded recombinant soluble GLPR1 and of a unique binding site in sGLIPR1, the invention features molecules that can bind to and/or otherwise inhibit the activity of GLIPR1 (i.e., GLIPR1 inhibitors). For example, GLIPR1 inhibitors may be proteins (e.g., antibodies), peptides, small molecules or nucleic acids (anti-sense, siRNA, etc.), which bind to GLIPR1 or a gene encoding a GLIPR1 protein and inhibits or reduces protein function or gene expression.

Inhibitors may bind GLIPR1 on the surface of cells, e.g., cancer cells or cells infected with a virus. For example, inhibitors may bind GLIPR1 in lipid rafts on the surface of cells. Binding of inhibitors to GLIPR1 may block or reduce the level of matrix metalloproteinase-2 secretion or cell proliferation, migration or spheroid formation Inhibitors may also downregulate cell surface GLIPR1 and/or induce AKT cleavage. Accordingly, GLIPR1 inhibitors may be administered to a subject alone or in conjunction with a pharmaceutically acceptable carrier, to treat or prevent a cancer, in which GLIPR1 contributes to tumor cell growth, or a viral infection, where GLIPR1 contributes to viral pathogenesis.

### 4. Pharmaceutical Compositions

Pharmaceutical compositions can comprise a GLIPR1 inhibitor and, optionally, a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition may be formulated to be compatible with an intended route of administration. Examples of routes of administration include parenteral, e.g., intravenous, intradermal, subcutaneous, oral (e.g., inhalation), transdermal (topical), transmucosal, vaginal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating a GLIPR1 inhibitor in an appropriate amount of a suitable solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery. Vaginal suppositories or foams for local mucosal delivery may also be prepared to block sexual transmission.

Inhibitors can be prepared with carriers that will protect it from rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers.

Oral or parenteral compositions may be formulated in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects. The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### 5. Therapeutic Uses

Based on their ability to inhibit cancer cell MMP-2 secretion, proliferation, invasion, migration and/or neurosphere formation, downregulate cell surface GLIPR1 and/or induce AKT cleavage, further featured are methods for treating or preventing in a subject a cancer, in which GLIPR1 contributes to tumor cell growth (e.g., glioma), comprising administering to the subject an effective amount of a GLIPR1 inhibitor, or a pharmaceutical preparation of the same. In addition, an effective amount of a GLIPR1 inhibitor, alone or in conjunction with a pharmaceutically acceptable carrier, may be administered to a subject to treat or prevent a viral infection where GLIPR1 contributes to viral pathogenesis. GLIPR1 inhibitors may also be useful for modulating the proliferation, survival and/or differentiation of cells in which GLIPR1 or AKT regulate cell proliferation, survival and/or differentiation.

The inventions described above are further illustrated by the following examples, which should not be construed as limiting. The contents of all references, patents and published patent applications cited throughout this application, as well as the Figures and the Sequence Listing, are incorporated herein by reference.

### EXAMPLES

### Example 1

### Structure Determination

A. Materials and Methods.

1. Structure Determination

Recombinant sGLIPR1 expression, purification, crystallization and data collection have been described (Bonafé, N. et al., (2010) Acta Crystallogr Sect F Struct Biol Crys Commun 66, 147-9). In addition to the reported conditions, crystals with the same morphology and similar cell constants are obtained using precipitant containing 0.17 M acetate, 0.085 M Tris-HCl pH 8.5, 25.5% PEG 4000 and 15% glycerol as well as replacing the acetate with comparable concentration of ammonium sulfate. Crystallographic data are shown in Table 1. Initial phases were obtained by MR with the program PHASER (McCoy, A. J. (2002). New applications of maximum likelihood and Bayesian statistics in macromolecular crystallography. Curr Opin Struct Biol 12, 670-3. McCoy, A. J. (2004). Liking likelihood. Acta Crystallogr D Biol Crystallogr 60, 2169-83. McCoy, A. J., Grosse-Kunstleve, R. W., Storini, L. C. & Read, R. J. (2005). Likelihood enhanced fast translation functions. Acta Crystallogr D Biol Crystallogr 61, 458-64. McCoy, A. J., Storini, L. C. & Read, R. J. (2004). Simple algorithm for a maximum-likelihood SAD function. Acta Crystallogr D Biol Crystallogr 60, 1220-8. Read, R. J. (2001). Pushing the boundaries of molecular replacement with maximum likelihood. Acta Crystallogr D Biol Crystallogr 57, 1373-82. Storini, L. C., McCoy, A. J. & Read, R. J. (2004). Likelihood-enhanced fast rotation functions. Acta Crystallogr D Biol Crystallogr 60, 432-8.) by using a truncated polyalanine search model containing only the alpha helices and strand regions of the CAP domain of stecrisp (pdb code 1RC9; Guo, M., Teng, M., Niu, L., Liu, Q., Huang, Q. & Hao, Q. (2005). Crystal structure of the cysteine-rich secretory protein stecrisp reveals that the cystein-rich domain has a K⁺ channel inhibitor-like fold. J Biol Chem 280, 12405-12.)). All MR attempts using multiple CAP search models containing loop regions failed. The initial MR solution resulted in one monomer per asymmetric unit, which, based on the volume of the unit cell being 26992 Å³, corresponds to a Matthews' coefficient of 3.11 Å³ Da⁻¹ (60% solvent). MR was followed by automatic model building using the CCP4 statistical protein chain tracing program BUCCANEER. (Cowtan, K. (2006). The Buccaneer software for automated model building. 1. Tracing protein chains. Acta Crystallogr D Biol Crystallogr 62, 1002-11. The structure was improved through iterative manual model building cycles using COOT (Emsley, P. “& Cowtan, K. (2004). Coot: model-building tools for molecular graphics. Acta Crystallogr D Biol Crystallogr 0, 2126-32.) followed by refinement using both PHENIX (Afonine, P. V., Grosse-Kunstleve, R. W. & Adams, P. (2005). The Phenix refinement framework. CCP4 Newsletter July, Contribution 8. Adams, P. D., Afonine, P. V., Bunkoczi, G., Chen, V. B., Davis, I. W., Echols, N., Headd, J. J., Hung, L. W., Kapral, G. J., Grosse-Kunstleve, R. W., McCoy, A. J., Moriarty, N. W., Oeffner, R., Read, R. J., Richardson, D. C., Richardson, J. S., Terwilliger, T. C. & Zwart, P. H. (2010). PHENIX: a comprehensive Python-based system for macromolecular structure solution. Acta Crystallogr D Biol Crystallogr 66, 213-21.) and REFMAC5 (Murshudov, G. N. (1997). Refinement of macromolecular structures by the maximum-likelihood method. Acta Crystallogr D Biol Crystallogr 53, 240-55. Murshudov, G. N., Vagin, A. A. Lebedev, A., Wilson, K. S. & Dodson, E. J. (1999). Efficient anisotropic refinement of macromolecular structures using FFT. Acta Crystallogr D Biol Crystallogr 55 (Pt 1), 247-55. Pannu, N. S., Murshudov, G. N., Dodson, E. J. & Read, R. J. (1998). Incorporation of prior phase information strengthens maximum-likelihood structure refinement. Acta Crystallogr D Biol Crystallogr 54, 1285-94.) with free-R (Brunger, A. T. (1992). The Free R Value: a Novel Statistical Quantity for Assessing the Accuracy of Crystal Structures. Nature 355, 472-474.) to yield a final model with statistics listed in Table 1.

TABLE 1
Statistics for data collection and model refinement

| Data | sGLIPR1 (3Q2U) | | Zn²⁺ complex (3Q2R) | |
|---|---|---|---|---|
| Space group | P2₁2₁2 | | P2₁2₁2 | |
| Unit-cell | a = 85.1, b = 79.5, | | a = 85.9, b = 79.7, | |
| parameters (Å) | c = 38.8, | | c = 38.8 | |
| Resolution limits (Å) | 29.0-1.85 | (1.95-1.85) | 27.8-2.0 | (2.1-2.0) |
| <I/σ(I)> | 20.1 | (3.2) | 7.9 | (2.5) |
| Number reflections | 279138 | (16237) | 108556 | (12118) |
| Number unique | 23229 | (3305) | 18698 | (2680) |
| reflections | | | | |
| Multiplicity | 12.0 | (4.9) | 5.8 | (4.5) |
| R_{merge}^{a} (%) | 9.3 | (49.6) | 17.9 | (62.1) |
| Completeness (%) | 99.9 | (99.4) | 99.4 | (98.1) |
| Refinement | | | | |
| R-cryst^{b}/R-free^{c} | 3.4/18.4 | (10.0/22.2) | 17.5/22.3 | (22.6/29.9) |

| | | |
|---|---|---|
| Correlation | | |
| coefficient | | |
| Fo-Fc | 0.965 | 0.925 |
| Fo-Fc (free) | 0.942 | 0.895 |
| Rms deviation | | |
| Bond lengths (Å) | 0.026 | 0.029 |
| Bond Angles (°) | 1.888 | 2.235 |
| Model composition | | |
| Monomers | 1 | 1 |
| Residues | 193 | 192 |
| Waters | 286 | 258 |
| Zn²⁺ | 0 | 1 |
| Ramachandran (%) | | |
| Preferred Regions | 96.34 | 94.2 |
| Outliers | 1.0 | 0.53 |

Values in parenthesis are for highest resolution shell
^{a}R_{merge} = ΣₕₖₗΣᵢ|Iᵢ(hkl) − [I(hkl)]|/ΣₕₖₗΣᵢIᵢ(hkl), where Iᵢ(hkl) and [I(hkl)] are the intensity of measurement of I and the mean intensity of the reflection with indices hkl, respectively.
^{b}R-cryst = Σ||Fo| − |Fc||/Σ|Fo| where Fo are observed and Fc are calculated structure factors amplitudes.
^{c}R-free set uses 5% of randomly chosen reflections.

The complex with Zn²⁺ was generated by soaking preformed crystals for 10 minutes with 0.2 mM zinc chloride in crystallization buffer, comprised of 0.085 M sodium cacodylate pH 6.5, 25.5% (w/v) PEG 8000, 0.17 M ammonium sulfate, 15% (w/v) glycerol. Longer soaks resulted in cracking of the crystals. Data from the Zn²⁺ soaked crystals were collected using a 4-circle kappa platform Xcalibur PX Ultra with a 165 mm diagonal Onyx CCD detector and a high brilliance sealed tube Enhance Ultra (Cu) X-ray source (Oxford Diffraction, Oxford UK), operating at 50 kV and 50 mA at a crystal-to-detector distance of 65 mm and exposure times of 150 seconds per 0.5° oscillations. The data sets were processed using the program Crysalis Pro (Oxford Diffraction). The structure was solved by MR using the structure of sGLIPR1 as the search model. The Zn² ion was located in the initial MR Fo-Fc electron density maps at 7 sigma contour levels. This was followed by iterative cycles of model building with COOT and structure refinement with REFMAC-5 and PHENIX to yield a model with statistics shown in Table 1.

2. Antibodies

Purified sGLIPR1 protein was used to immunize rabbits following standard immunization principles. Likewise, the membrane fraction of glioblastoma U87MG cells was prepared using previously developed methods (Toy, et al., (2005) Correlation of tumor phenotype with c-fms proto-oncogene expression in an in vivo intraperitoneal model for experimental human breast cancer metastasis. Clin Exp Metastasis 22, 1-9.) and used for rabbit immunizations. To affinity purify anti-GLIPR1 antibodies from the rabbit antisera, an affinity column was prepared by immobilizing the sGLIPR1 antigen to a beaded agarose support (AminoLink Immobilization, Pierce Biotechnology). Peptide GLIPR1₇₅₋₉₅ (CQFSHNTRLKPPHKLHPNFTS, SEQ ID NO: 27), spanning residues unique to GLIPR1, was conjugated to KLH and used to immunize rabbits. Anti-GLIPR1₇₅₋₉₅ IgGs were isolated from the antiserum using a HiTrap Protein-G HP column, following the manufacturer's recommendations (GE Healthcare) and used as a control antibody. Freund's adjuvant was used in all immunization protocols. Normal rabbit IgG was purchased from Sigma Aldrich.

3. Epitope Mapping

To identify GLIPR1 linear epitopes targeted by the anti-GLIPR1 antibodies, overlapping 16-mer peptides (overlap of 12, offset of 4) spanning the sGLIPR1 sequence (amino-acid residues 20-220) were synthesized and attached to a solid support (Pepset peptide array; Mimotopes). Each anti-GLIPR1 antibody was examined for binding to the peptide array in an ELISA format (and compared with the company's positive and negative controls), according to the manufacturers' protocol. Antibody preparations include anti-sGLIPR1 and anti-native GLIPR1 that inhibit U87 glioma cell growth, and normal rabbit IgG and anti-GLIPR1₇₅₋₉₅ negative control antibodies that do not inhibit U87 cell growth.

B. Results and Discussion.

1. Structure Determination and Model Quality

Human GLIPR1 (NCBI accession no. NP_{—}006842) is naturally synthesized as a 255 amino acid precursor with a signal peptide and a transmembrane domain that localizes the mature protein to the cell membrane in glioblastoma cells. The first 21 residues make up the signal peptide while C-terminal residues form the membrane spanning domain. The recombinant sGLIPR1 protein consists of the amino acids 22-220 of the human GLIPR1 with a P. pastoris signal peptide and the linker amino acid sequence EAEAEF (SEQ ID NO: 29) added to the N-terminus by the cloning procedures. The recombinant protein was truncated before the C-terminal GLIPR1 transmembrane domain and no purification tag was added. The first residue of the mature peptide in the structural model is residue number 22. Purified recombinant sGLIPR1 is a monomer showing a molecular mass greater than the expected theoretical molecular weight of 23,295 Da. Electro-spray mass spectrometry analyses and deglycosylation experiments suggested the presence of glycosylation. An examination of the omit 2FoFc electron density maps reveal that there is sufficient electron density in the proximity of N92 to confirm glycosylation. These results are in agreement with the single predicted N-linked glycosylation site at residue N92.

Of the mature polypeptide of sGLIPR1, 193 amino acids have unambiguous main chain density in REFMAC-5 weighted 2FoFc maps contoured at 1 sigma. The first residue A22 only has visible electron density at 0.8 sigma contoured maps. However, the C-terminus residues (KRYYS, SEQ ID NO: 28) and plasmid-incorporated linker sequence (EAEAEF, SEQ ID NO: 29) are disordered and cannot be modeled. The only additional disordered regions at 0.8 sigma contoured maps are the side chains of residues that are solvent exposed and lacking substantial contacts with other amino-acids, notably K84, K133, N197, N198 and N204. The structure has high solvent content (60%), but there are also regions of density that were not modeled, which are clearly not water molecules. Some could be modeled as glycerol molecules but others had irregular shape and were not modeled. The coordinates and structure factors for the native and zinc chloride soaked structures have been deposited with the RCSB Protein Data Bank with accession codes 3Q2U and 3Q2R respectively.

2. Structure of Uncomplexed sGLIPR1

The overall structure of sGLIPR1 consists of an N-terminal loop, followed directly by the CAP domain and a cysteine-rich C-terminal region (FIGS. 1 and 2). The tertiary structure of the conserved core CAP motif of sGLIPR1 is a 3-stranded anti-parallel β sheet sandwiched between two layers of α helices. As is typical of all CAP domains, one α helical layer is composed of two parallel α helices (α1 and α3) while the other has a solitary α helix (α2). While α1 and α2 are regular alpha helices, α3 contains an alpha helix that turns into a 3₁₀-helix, reminiscent of the Na-ASP-2 structure. The CAP anti-parallel β sheet is formed by the 3 longest β strands; β2 (G97 through S104), β5 (K148 through C156) and β6 (G168 through G176). Directly following the CAP domain are the residues that likely serve as an anchor between the soluble CAP domain and the trans-membrane domain. This C-terminal region is structurally similar to those of other CAPs, notably Na-ASP-2 and all reported snake venom CRISP structures. It consists of a 2-stranded β sheet linked by disulfide bonds 4 (C192/C201) and 5 (C195/C206) and terminates in a short two turn helix (FIGS. 1 and 2). All cysteines in sGLIPR1 are involved in disulfide bonds except for C37. In total, there are five disulfide bonds in sGLIPR1 (FIGS. 1 and 2).

Uniquely, sGLIPR1 has longer, differently positioned and more protrusive loops linking the secondary structure regions than any of the previously reported CAP structures (FIGS. 1, 2 and 4). The unique lengths and orientations of these loops may explain why phasing by MR failed when the loops were not removed from the initial search model. Of the 193 residues in the sGLIPR1 model, 54.4% are in loop or turn regions, including Ala22-Asn30 (SEQ ID NO: 11), Glu47-Met58 (SEQ ID NO: 13), Ser73-Leu96 (SEQ ID NO: 15), Val105-Ser109 (SEQ ID NO: 17), Phe126-Arg129 (SEQ ID NO: 19), Pro157-Asn167 (SEQ ID NO: 21), Gly176-Asp199 (SEQ ID NO: 23) and Leu202-Asn204 (SEQ ID NO: 25) (See FIGS. 1 and 2). These are mostly on the surface of the structure (FIG. 1). These non-homologous regions made significant contributions to the initial MR search model, which was also observed in structural studies of other CAP proteins. The unique lengths and orientations of these loop/turn regions are likely to explain why phasing by MR failed when the loops were not removed from the initial search model. These structurally nonhomologous regions made significant contributions to the initial MR search model, as was observed in structural studies of hookworm CAP proteins (Asojo, et al., (2005). J Mol Biol 346, 801-14. Asojo. (2011). Acta Crystallogr D Biol Crystallogr 67:455-62.). Additionally these regions are quite different than predicted in the homology model of sGLIPR1 (Szyperski, T., et. al.,. (1998) Proc Natl Acad Sci USA 95, 2262-6). The length of the helices and strands, and the orientation and location of loops could not be predicted based on any previous CAP structures, despite the alignment of the conserved core motif.

3. Comparison with Representative CAP Structures

Comparison of the sGLIPR1 structure with those of representative member of CAP protein subfamilies, Na-ASP-2 (1U53) (Asojo, et al., (2005) J. Mol Biol 346: 801-14), GAPR-1 (1SMB) (Serrano, R. L., et al., (2004) J. Mol Biol 339:173-83), Ves v5 (1QNX) (Henriksen, A. et. al., (2001) Proteins 45:438-48), stecrisp (1RC9) (Guo, M et al., (2005) J. Biol Chem 280: 12405-12), reveals that the core secondary structure elements of the α-β-α sandwich in the CAP domain are conserved, albeit having different lengths (FIGS. 1 and 2). Additionally 3 of 5 disulfide bonds of sGLIPR1 are conserved in both Na-ASP-2 and stecrisp (FIGS. 1 and 2). The importance of the disulfides is tempered by the observation that the CAP core of crystallized recombinant GAPR-1 is stable without any cysteines and disulfide bonds. The loop regions in sGLIPR1 reveal the greatest differences when compared to other CAP sequences, whereas the two signature PROSITE (http://www.expasy.org/cgi-bin/prosite/PSScan.cgi) CRISP motifs are largely conserved (FIG. 2). Apart from the loop regions, the C- and N-terminal regions of these representative CAP structures have the greatest variation. Interestingly, the overall structure of sGLIPR1 appears to be more similar to that of Na-ASP-2 than to the other representative CAP proteins (FIG. 1).

Despite the tertiary structure similarity, the charge distribution on sGLIPR1 and Na-ASP-2 is quite different. The charge distribution on Na-ASP-2 is almost completely separated with one side being mostly electronegative whereas the other side was mostly electropositive (Asojo, O. A. et al., (2005) J Mol Biol 346:801-14). The charge distribution on sGLIPR1 is not as separated but is more clustered than that of Na-ASP-2 and there does not appear to be any consensus in the charge distribution among representative CAP structures (FIG. 1).

4. Putative CAP Binding Cavity

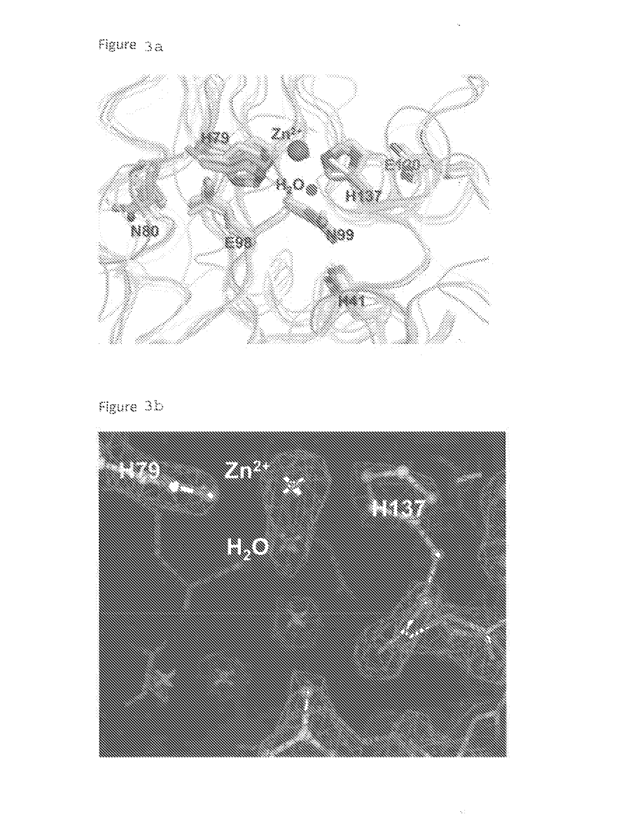

Despite the differences in overall charge distribution, CAP proteins are characterized by a large central charged cavity containing key conserved charged residues. Key among these residues in GLIPR1 is H137 that is part of the PROSITE recognized CRISP signature motif 1: 136 GHYTQVVWADS146 (SEQ ID NO: 30). Additionally, GLIPR1 contains CRISP family signature 2: 170HFICNYGPGGNY181 (SEQ ID NO: 31). The conserved residues that lie in the putative binding cavity of sGLIPR1 align well with those in representative CAP structures (FIG. 3). These residues were previously hypothesized to be part of the active site residues that could form the catalytic triad of a putative serine protease (Gibbs, G. M., et al., (2008) Endocr Rev 29: 865-97; Asojo, O. A. et al., (2005) J Mol Biol 346:801-14). In most of the reported CAP structures there is a serine which lies in the cavity, but it is oriented such that it is incapable of forming a typical serine protease catalytic triad with the proximal histidine. Uniquely, sGLIPR1 lacks this postulated catalytic serine, in its place there is N80, which is likewise oriented away from the conserved H79 (FIG. 3). The crystallographic dimers of sGLIPR1 are incapable of forming the catalytic triad of a conventional serine protease as is the case with all other CAP structures currently in the Protein Data Bank. Unsurprisingly, no proteolytic activity has been detected with sGLIPR1 and it does not have any significant sequence similarity to any known peptidase in the MEROPS database (http://merops.sanger.ac.uk/).

Another role of this conserved cavity was suggested by studies which revealed it as the major Zn²⁺ binding site in the Zn²⁺ and heparin-sulfate dependent mechanisms of inflammatory modulation by the cobra CRISP natrin (3MZ8) Wang, Y. L et al., (2005) J Biol Chem 285: 37872-83). The two conserved histidines that directly coordinate the Zn²⁺ have similar positions and orientations similar in the sGLIPR1 structure (FIG. 3). Cobra CRISP natrin has an additional serine, in close proximity to the cavity. This serine is not conserved in sGLIPR1 or other mammalian CAPs—instead sGLIPR1 has N99 that aligns well with the serine. The structure of Zn²⁺ bound pseudecin (2EPF) Suzuki, N., et al. (2008) Acta Crystallogr D Biol Crystallogr 64: 1034-42) reveals that a snake-venom CRISP with an asparagine instead of serine has the ability to form the same network of bonds (FIG. 3), thus it appeared quite plausible that Zn²⁺ will bind in a very similar fashion in sGLIPR 1.

To investigate if Zn²⁺ binds to sGLIPR1, preformed crystals of sGLIPR1 were soaked with 0.15 mM ZnCl₂ and the structure of the complex was determined. The electron density maps of crystals soaked with zinc chloride suggest that Zn²⁺ binds to sGLIPR1 (FIG. 3). Furthermore, the putative binding cavity of sGLIPR1 forms a similar network of bonds with Zn²⁺ as was observed in natrin and pseudecin. Since CAP proteins are often produced in conditions involving host immune responses, it is plausible that the conserved cavities in different CAP proteins have similar roles in Zn²⁺ dependent modulation of inflammation. The central cavity is exposed in all CAP proteins and accessible to inflammatory agents and other molecules that may bind to these proteins.

5. Antibody Linear Epitope Mapping

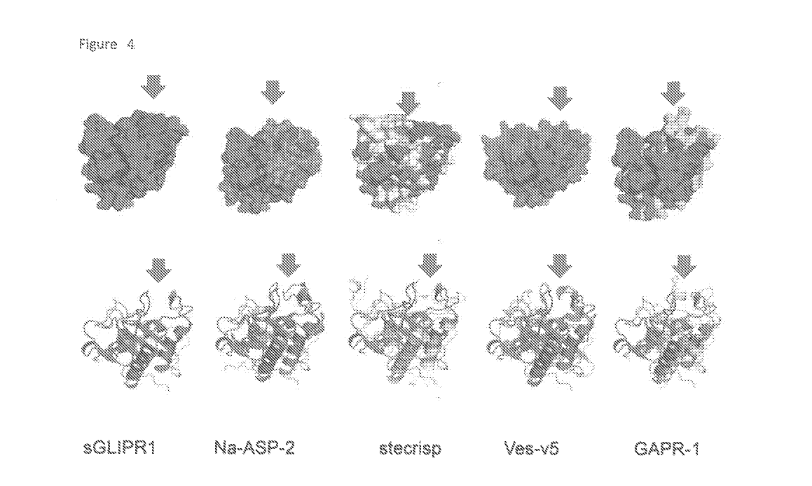

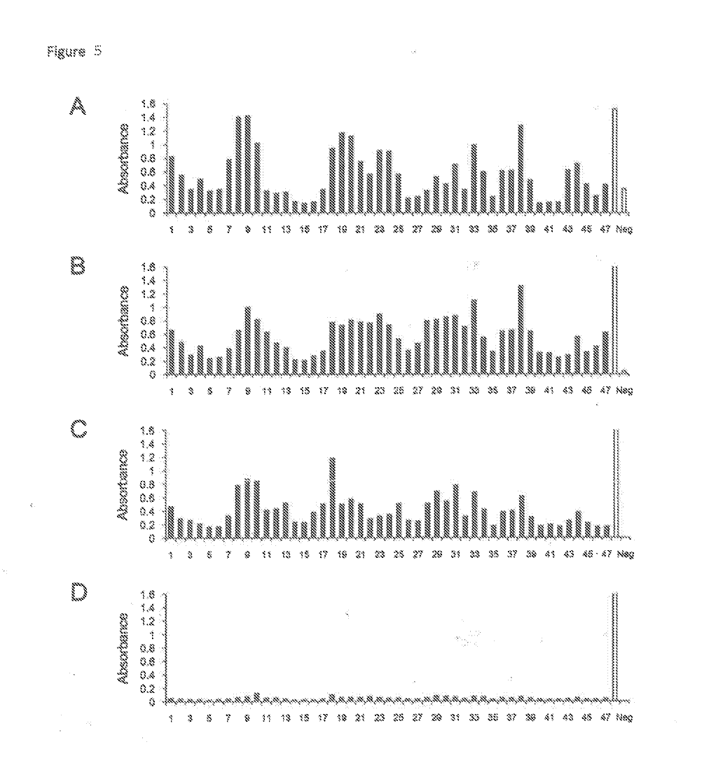

Rabbit anti-sGLIPR1 antibodies were produced as described below. Anti-sGLIPR1 antibody was affinity-purified with immobilized sGLIPR1 from antisera produced by immunizing rabbits with purified recombinant sGLIPR1. Anti-native GLIPR1 antibody was affinity-purified with immobilized sGLIPR1 from antisera produced by immunizing rabbits with a membrane preparation enriched in native GLIPR1 from U87 glioma cells. These antibodies, which inhibit U87 glioma cell proliferation, were tested with negative control antibodies for binding to a series of 16-mer peptides with 12 residue overlaps that span the entire sGLIPR1 sequence. Of these 16-mer peptides, peptide 38 (SEQ ID NO: 1), had the strongest reactivity uniquely with the anti-sGLIPR1 and the anti-native GLIPR1 antibodies that inhibit U87 glioma cell growth, and not with negative control rabbit antibodies (FIG. 5). Interestingly, peptides 37 (166-SNGAHFICNYGPGGNY-181, SEQ ID NO: 32) and 39 (174-NYGPGGNYPTWPYKRG-189, SEQ ID NO: 33) that overlap with peptide 38 also positively reacted with absorbance approximately half of the peptide 38 absorbance. Peptide 38, with peak antibody reactivity, spans much of the sGLIPR1136 strand and the entire conserved PROSITE CRISP2 motif, and extends into a cleft adjacent to the CRISP2 motif that is framed by a loop unique to sGLIPR1 (FIGS. 2 and 4). This unique cleft is obstructed by main chain atoms in other CAP protein structures, whereas in GLIPR1 this cleft contains antibody binding epitopes (FIG. 4). The extent of closure is evident in the reduction of the equivalent distance from the Cα of R114 to Cα T168 from 7.7 Å in sGLIPR1 to 4.3 Å (stecrisp), 3.8 Å (yes v5), 3.2 Å (Na-ASP-2) and 2.0 Å (GAPR-1). This reduction of the cleft accessibility may be sufficient to limit access of antibodies and other potential binding partners to this motif in the other CAP proteins relative to sGLIPR1.

### Example 2

### Antibodies to GLIPR1 Inhibit Cancer Cell MMP-2 Secretion, Proliferation, Migration, Neurosphere Formation, Downregulate Cell Surface GLIPR1 and Induce AKT Cleavage

A. Materials and Methods.

1. Cell Lines, Culture, Protein and RNA Extraction and Analysis

All human cell lines were obtained from the American Tissue Culture Collection (ATCC). Glioblastoma (U87, U118 and A172), neuroblastoma (SKNF-1) and epidermoid carcinoma (A431) cell lines were propagated in Dulbecco's modified Eagle medium (DMEM high glucose, Invitrogen). Melanoma (WN266), breast carcinoma (MCF-7), neuroblastoma (Neuro-2A) cell lines and normal foreskin fibroblasts (BJ) were cultured in Eagle's Minimum Essential Medium (EMEM, ATCC). The breast carcinoma cell line BT474 was cultured in Hybricare medium (ATCC). For all cell lines, the culture medium was supplemented with 10% fetal bovine serum, 2 mmol/L glutamine, 100 units/mL penicillin, and 0.1 mg/mL streptomycin and maintained in a humidified incubator at 37 C with 5% CO₂.

Protein Extracts:

Cells were gently scraped from their flasks and washed twice with PBS. For total protein extracts, cells were resuspended in 25 mM Hepes pH 7.9, 150 mM NaCl, 2 mM EDTA, 1 mM DTT, 1× protease inhibitor (Roche) and 0.1% NP-40 and lysed on ice for 1-2 hours. For membrane and cytoplasmic protein extracts, cells were resuspended in hypotonic 10 mM Tris pH 7.4, 10 mM NaCl, 3 mM MgCl₂, and protease inhibitors, kept on ice for 10 minutes, and then broken using a glass dounce homogenizer (Kontes Glass Co.). Centrifugation at 3000 rpm for 5 minutes isolated nuclear pellets and supernatants. Supernatants were centrifuged at 14,000 rpm for 15 minutes to recover the membrane fraction (pellet) and the cytoplasmic fraction (supernatant).

SDS-PAGE and Immunoblot Analyses:

Protein samples were size fractionated in Tris-Glycine buffer by SDS-PAGE. Some conditions required reducing agents (DTT), some did not (non-reducing). After electrophoresis, gels were either Coomassie Blue-stained, silver-stained (ProteoSilver, Sigma), or processed for immunoblot analysis, according to standard procedures. The anti-actin antibody (clone AC-15, Sigma) was used based on the manufacturer's recommendations. Anti-GLIPR₇₅₋₉₅ peptide and sGLIPR1 antisera were used at 1:10,000 dilutions. Immunoglobulins (IgGs) isolated from GLIPR1₇₅₋₉₅ peptide antiserum using a HiTrap Protein G HP affinity column (GE Healthcare), according to the manufacturer's protocol, were used. Antibodies obtained from Cell Signaling Technology were used at the following dilutions: anti-AKT, 1:500; anti-phospho-AKT (ser 473), 1:300; anti-procaspase 3, 1:200; and anti-Bcl2, 1:800.

Reverse Transcription-PCR.

Total RNA was extracted from cell pellets by RNeasy (Qiagen), according to the manufacturer's protocol. Five μg of total RNA was transcribed into cDNA using a AffinityScript cDNA synthesis kit (Stratagene). In a semi-quantitative PCR using Taq DNA polymerase (Roche), relative levels of GLIPR1 mRNA were estimated by comparison with actin mRNA levels. Amplification conditions were 94° C. for 1 minute, then 35 cycles of: 94° C. for 30 seconds; 57° C. for 30 seconds; 68° C. for 30 seconds; and finally 72° C. for 5 minutes. PCR products were size fractionated by electrophoresis in 2% agarose gels and stained with ethidium bromide. The following primers were used. GLIPR1, forward primer: 5′-GATGGTTTCTTTTGTCTCCA (SEQ ID NO: 34); reverse primer: 5′-GTTAACACAGAGATTGTCCA (SEQ ID NO: 35). Actin, forward primer: 5′-GCTCGTCGTCGACAACGGCTC (SEQ ID NO: 36); reverse primer: 5′-CAAACATGATCTGGGTCATCTTCTC (SEQ ID NO: 37).

Antibody Production:

Rabbit polyclonal anti-GLIPR₇₅₋₉₅ peptide antiserum was produced by Multiple Peptide Systems (San Diego, Calif.). The following peptide, CQFSHNTRLKPPHKLHPNFTS (SEQ ID NO: 38), was synthesized, conjugated to KLH via the N-terminal cysteine, by MBS (m-Maleimidobenzoyl-N-hydroxysuccinimide ester, Pierce) crosslinking and used to immunize rabbits, according to a standard protocol. Rabbit polyclonal anti-sGLIPR1 antibody was produced by Cocalico Biologicals, Inc., (Reamstown, Pa.), also according to a standard immunization protocol. New Zealand white rabbits were immunized with 50 μg of recombinant sGLIPR1 antigen/injection emulsified in Freund's adjuvant, and boosted at day 14, 21 and 35. Pre-immune serum was collected for each rabbit as negative controls. To specifically isolate anti-GLIPR1 antibodies from the antiserum, the three rabbit antisera were pooled and applied to an affinity column made of sGLIPR1 immobilized on agarose beads (AminoLink immobilization kit, Pierce, Ill.). After a series of washes, polyclonal anti-GLIPR1 IgGs were eluted with 0.1M glycine buffer pH 2.7, neutralized to pH 7, buffer exchanged and concentrated (final buffer: PBS/50% glycerol) to 2 mg/mL. One rabbit was also immunized at Cocalico Biologicals, Inc., with cell membrane preparation of the glioblastoma cell line U118 (100 μg/injection). Anti-native GLIPR1 antibodies were further affinity-purified, using the sGLIPR1 affinity column and method described above.

Enzyme-Linked Immunosorbent Assay (ELISA):

A standard ELISA method in a 96-well plate format (Bonafé, N., et al., (2009) Vaccine 27: p. 213-22) was used to assess antibody binding to sGLIPR. In brief, the assay consists of coating the sGLIPR1 at 100 ng/well in carbonate buffer, overnight at 4° C. Plates were then washed with PBS/Tween, blocked in PBS/Tween-5% dry milk, and incubated for one hour at room temperature with diluted antiserum or purified antibody. The plates were washed three more times in PBS/Tween and incubated with secondary alkaline-phosphatase-labeled anti-rabbit antibody (Sigma; 1/3000) for one hour at room temperature before the alkaline phosphotase signal was detected with a solution of p-nitrophenyl phosphate (Sigma).

Immunoprecipitation:

The Pierce Co-Immunoprecipitation Kit was used to demonstrate that anti-sGLIPR1 antibodies bind native GLIPR1 in fresh cell lysates. Glioblastoma cells (U87) and control melanoma cells (WN266) were grown in flasks, gently washed and scraped from their cell culture support before lysis in 25 mM Tris, pH 7.4, 150 mM NaCl, 1 mM EDTA, 1% NP-40, 5% glycerol for 30 minutes on ice. 500 μl of buffer was used to disrupt 50 mg of wet cell pellet. Cell debris was removed by centrifugation at 13,000×g for 10 minutes. The lysates were applied to anti-sGLIPR1 antibodies covalently immobilized to AminoLink Plus coupling resin, provided in the kit, according to the Pierce's instructions.

Isolation of Lipid Raft/Caveolae Cell Fraction:

All procedures were performed on ice using a Caveolae/Rafts Isolation kit (Sigma), following the manufacturer's instructions. After cell collection, 1 mL of lysis buffer containing 1% Triton X-100 was added and incubated on ice for 30 minutes. The density gradient was made of five layers of Optiprep from 35% to 0%. The lower layer (35% OptiPrep) contained the cell lysate. The density gradient was centrifuged at 200,000×g (70.1 Ti rotor, Beckman Coulter) for 4 h at 4° C. One mL fractions were carefully collected from top to bottom of the centrifugation tube, and each subjected to TCA precipitation. Protein pellets were resuspended in 100 μL Laemmli buffer and 10 μL were analyzed by SDS-PAGE. Lipid raft/caveolae fractions were tracked by the enrichment of the cholesterol-binding protein caveolin-1.

Immunofluorescence Assays (IFA):

Adherent cells were grown on culture slides (BD-Falcon) for 12-24 hours and fixed with 2% paraformaldehyde in PBS for 10 minutes at RT. U87 spheroids were transferred from a culture flask or a 96-well plate into a microtube to be processed and finally mounted onto a culture slide. Briefly, cells were washed in PBS, blocked with 2% FBS/PBS for 30 minutes at RT, and incubated for 1 hour with primary antibodies diluted in 2% FBS/PBS. Following incubation with FITC-labeled anti-rabbit conjugate (Sigma) for 45 minutes at RT, cell nuclei were sometimes stained with 20 μg/mL propidium iodide for 5 minutes at RT. Samples were finally mounted in anti-fade solution (Vectashield, Vector) and fluorescent images captured using a Zeiss Axiovert 200M inverted microscope or confocal microscopy.

2. Cell-Based Assays.

Cell Adhesion:

Serum-starved glioma cells (grown 12-16 h in serum-free growth medium supplemented with 0.1% BSA) were seeded onto a fibronectin-coated cell culture plate (96-well plate format, Becton Dickinson) for 2 hours at 37° C., in absence or presence of purified IgGs. Following PBS washes, and fixation in 4% paraformaldehyde for 15 minutes, the attached cells were stained with a crystal violet solution for 10 minutes, washed and dried. Relative quantitation of cell adhesion (treated/non-treated/control) was measured by addition of a 2% SDS solution to the wells, and reading at 550 nm in a Biotek platereader.

MMP-2 Secretion:

Serum-starved cells were maintained in culture at 37° C. for 48 h, in absence or presence of anti-GLIPR1 IgGs (100 μg/mL) Conditioned media were collected and tested with the EnzoLyte™ 490 MMP-2 Assay Kit (AnaSpec, San Jose, Calif.), according to the manufacturer's recommendations. The assay is designed to detect MMP-2 proteolytic activity in a variety of samples using an EDANS/DABCYL fluorescence resonance energy transfer peptide. Fluorescence increases upon active MMP-2 cleavage of the FRET peptide. The fluorescence reading from the substrate control wells represented the background fluorescence; it was subtracted from the reading of the other wells to obtain the relative fluorescence units (RFU). For gelatin zymographic analysis, conditioned media were concentrated 10 times using an Amicon Ultra centrifugal filter device (10 k). Each concentrated condition medium was subjected to SDS-PAGE containing 0.1% gelatin. After electrophoresis, the gels were washed with Triton-X100 to remove SDS and incubated for 24-48 h at 37° C. in buffer containing 5 mM CaCl₂ and finally stained with Coomassie Brilliant Blue. Proteolytic activity is usually visualized as clear bands, zones of gelatin degradation, against the blue background of stained gelatin.

Transwell Cell Migration/Invasion Assay:

Cell lines were tested for their ability to invade through a human extracellular matrix in Matrigel™ invasion assays (BD Biosciences, Mass.). Serum-starved cells were seeded in 24-well chambers on a 8-μm pore polycarbonate filter coated with an uniform layer of Matrigel™ that serves as an authentic reconstituted basement membrane, providing a true barrier to non-invasive cells while presenting an appropriate extracellular protein structure to study invasion in vitro. The lower compartment contains growth medium with 0.5% FBS as chemo-attractants. Following incubation for 48 at 37° C., quantitation of cells in the lower compartment was achieved by fixing and staining the cells that have invaded (DiffQuik™). Migration of the cells through a non-matrigel coated control membrane provided a measure of cell migration.

Cell Proliferation:

The CellTiter 96® AQᵤₑₒᵤₛ One Solution Cell Proliferation Assay (Promega) is a colorimetric method for determining the number of viable cells in proliferation, cytotoxicity or chemosensitivity assays. The main reagent contains a tetrazolium compound [3-(4,5-dimethylthiazol-2-yl)-5-(3-carboxymethoxyphenyl)-2-(4-sulfophenyl)-2H-tetrazolium; MTS] and an electron coupling reagent (phenazine ethosulfate; PES). PES has enhanced chemical stability, which allows it to be combined with MTS to form a stable solution. Assays are performed by adding a small amount of the CellTiter 96® AQᵤₑₒᵤₛ One Solution Reagent directly to culture wells, incubating for 1-4 hours and then recording absorbance at 490 nm with a 96-well plate reader. The quantity of formazan product as measured by the amount of 490 nm absorbance is directly proportional to the number of living cells in culture.

Cytotoxicity:

The CytoTox-ONE™ Homogeneous Membrane Integrity Assay (Promega) is a fluorometric method for estimating the number of nonviable cells present in multiwell plates. The assay rapidly measures the release of lactate dehydrogenase (LDH) from cells with a damaged membrane. LDH released into the culture medium is measured with a 10-minute coupled enzymatic assay that results in the conversion of resazurin into a fluorescent resorufin product. The amount of fluorescence produced is proportional to the number of lysed cells using a 96- or 384-well format.

Spheroid (Neurosphere) Formation:

Cells grown in presence of serum were trypsinized, washed with PBS twice and resuspended in D-MEM/F12 supplemented with LIF, bFGF, EGF and B27 stem cell culture supplements. Upon 3-7 days of culture stimulating stem cell growth, spheroids had formed, and continued growing. Normal rabbit IgGs (100 μg/mL, Sigma), were used as control.

B. Results and Discussion.

Antibodies to GLIPR1 Inhibit Glioblastoma Cell MMP-2 Secretion.

RT-PCR and Western blot analyses corroborated previous findings from others that the U87 and A172 glioma cells over-expressed and moderately-expressed GLIPR1, respectively, while the SKNF1 neuroblastoma cells did not express detectable GLIPR1. In addition, MMP-2 protease activity secreted by these cells into the culture medium was confirmed to correlate with the level of GLIPR1 expression. With matrix metalloproteases (MMPs) playing important roles in the digestion of extracellular matrix and in the invasion of tumor cells, and particularly MMP-2 in glioma, anti-sGLIPR1 antibodies were examined for possible effects on active MMP-2 secretion by the U87 glioma cell line at 50 μg/mL. The antibodies showed significant inhibition (t-Test: Two-Sample Assuming Equal Variances P value 0.0135) of MMP-2 secreted into the culture supernatants. Under the same conditions, anti-peptide₇₅₋₉₅ IgGs had no effect on MMP-2 secretion; A172 cells were not sensitive to anti-GLIPR1 IgG treatment.

Antibodies to GLIPR1 Inhibit Glioblastoma Cell Migration.

Glioblastoma U87 cells were further assessed for inhibition by anti-GLIPR1 antibodies in an assay measuring cell migration and invasion. Cell invasion through inserts coated with a Matrigel membrane was measured after 48 h, and cell migration was evaluated using control transwell inserts without Matrigel. Compared to normal rabbit IgGs used as negative control, a significant effect of the anti-GLIPR1 antibodies was observed on cell migration and invasion with 100% inhibition reached at 50 μg/mL. Although the experiment does not differentiate whether inhibition of cell invasion was due to inhibition of cell migration or not, these data confirm previous findings that GLIPR1 plays an important role in glioma cell migration/invasion. Interestingly, when the antibodies were then examined for possible effects on cell adhesion, much larger quantities of reagent were used and resulted in only marginal inhibition of U87 adhesion (data not shown).

Antibodies to GLIPR1 Inhibit Cancer Cell Proliferation.

Glioblastoma cells, in addition to being invasive to the surrounding normal brain tissue, are also extremely proliferative, and GLIPR1 has previously been implicated in cell growth regulation. Whether the anti-GLIPR1 antibodies had any effect on the growth of U87 cells was therefore investigated. The first experiments indicated a highly significant inhibition of glioma cell growth that needed further scrutiny for specificity. To identify appropriate control cell lines for the next experiments, the GLIPR1 expression was estimated in a series of cell lines by RT-PCR, setting up a high number of PCR cycles to segregate between positive or negative cell lines. Relative to U87 cells that over-express glipr1 mRNA, it was found that epithelial carcinoma cells A431 were also high expressers, and that melanoma cells WN266 were also positive although low expressers. Breast cancer MCF7 and neuroblastoma Neuro2 were both found to have low or undetectable glipr1 expression. In an established proliferation assay, a set number of cells were initially seeded and left to proliferate as adherent cells for 72 hours, at which time the number of viable cells was quantified. Both anti-sGLIPR1 and the anti-native GLIPR1 antibodies were efficient at inhibiting proliferation of U87, A431 and WN 266 cell lines that express high to moderate levels of glipr1, while they had insignificant effect on MCF7 cells with low glipr1 expression.

Many key pathways are in play in the development of glioblastoma multiforme, not all known or well understood. It had been suggested that growth of cancer cell lines, including U87 cells, under stem cell-like conditions has the potential to unveil therapeutic targets that contribute to the growth of cancer stem cells. The anti-sGLIPR1 antibody was tested for inhibition of the glioblastoma cancer stem cell phenotype. Adherent U87 cells were trypsinized and resuspended in serum-free DMEM-12 medium, supplemented with growth factors promoting stem cell propagation. Generally, U87 cells formed spheroids upon 3-5 days of culture and those spheroid structures continued to grow if left in the medium for a few more days. Addition of saturating concentrations of anti-sGLIPR1 antibodies (ranging from 20 to 100 μg/mL for 2,000 to 10,000 cells seeded) to the serum-free culture medium resulted in complete inhibition of spheroid formation. The same results were obtained with the anti-native GLIPR1 antibodies. Cells in suspension in the serum-free medium defined above were also assessed in the semi-quantitative proliferation assay, where 10,000 cells were allowed to grow for 72 h, at which time cell growth was measured, as described above. In the control group (normal rabbit IgG; 100 μg/mL), cells grew 3.5-fold, while 20 μg/mL of anti-sGLIPR1 or anti-native GLIPR1 antibodies partially inhibited cell proliferation, and 50 to 100 μg/mL achieved nearly complete growth inhibition.

Antibodies to GLIPR1 Downregulate Cell Surface GLIPR1 and Induce AKT Cleavage.

The silencing of glipr1 expression was previously shown to decrease cell proliferation and induce cell apoptosis in glioma cells. Conversely, forced glipr1 overexpression caused increased glioma cell growth and overall resistance to apoptosis that correlated with increased apoptosis-related protein Bcl2 expression. To delineate the possible mechanisms involved in the inhibition of U87 growth upon anti-GLIPR1 antibody treatment, the expression of Bcl2, procaspase3 and AKT was examined, as was the relative actin level in western blot analyses. Unexpectedly, in U87 cells, treatment with antibodies for 48 h did not affect either the Bcl2 expression level or that of procaspase 3. Instead, cleavage of AKT and downregulation of GLIPR1 was detected. To understand better the mechanism of action, the level of expression of AKT and GLIPR1 were measured at 4, 24 and 48 h of treatment, and it was confirmed that these events started only a few hours after antibody treatment.

The practice of the present invention may employ, unless otherwise indicated, conventional techniques of cell biology, cell culture, molecular biology, transgenic biology, microbiology, recombinant DNA, and immunology, which are within the skill of the art. Such techniques are described in the literature.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A composition, which binds to a polypeptide selected from the group consisting of SEQ ID NOs: 1, 11, 13, 15, 17, 19, 21, 23 and 25.

2. A composition of claim 1, which is a protein or a peptide.

3. A composition of claim 1, which is an antibody.

4. A composition of claim 1, which is an inhibitor of GLIPR1

5. A pharmaceutical composition comprising an effective amount of a composition of claim 2 and a pharmaceutically acceptable carrier.

6. A composition of claim 1 conjugated to diagnostic, antiviral or anti-cancer agent.

7. A method of inhibiting GLIPR1 activity in a cell comprising contacting the cell with a composition of claim 2.

8. A method of claim 7, wherein the inhibitor binds GLIPR1 on the surface of cells.

9. A method of claim 7, wherein the inhibitor binds GLIPR1 in lipid rafts on the surface of cells.

10. A method claim 7, wherein the cell is a cancer cell or a virally infected cell.

11. A method of claim 7, wherein GLIPR1 or AKT contributes to the cancer cell's growth.

12. A method of claim 7, wherein GLIPR1 contributes to viral pathogenesis.

13. A method of claim 7, wherein the inhibitor reduces matrix metalloproteinase-2 secretion.

14. A method of claim 7, wherein the inhibitor reduces cell proliferation, migration or spheroid formation.

15. A method of claim 7, wherein the inhibitor downregulates cell surface GLIPR1.

16. A method of claim 7, wherein the inhibitor induces AKT cleavage.

17. A method for treating or preventing a cancer in a subject, comprising administering to the subject a pharmaceutical composition of claim 5.

18. A method of claim 17, wherein GLIPR1 or AKT contributes to the cancer cell's growth.

19. A method for treating or preventing a viral infection in a subject, comprising administering to the subject a pharmaceutical composition of claim 5.

20. A method of claim 19, wherein GLIPR1 contributes to viral pathogenesis.
